(19) United States
(12) Patent Application Publication
(10) Pub. No.: US 2023/0130379 A1
(43) Pub. Date: Apr. 27, 2023

(54) METHODS AND APPARATUS FOR OCCLUDING THE LEFT ATRIAL APPENDAGE

(71) Applicant: SQUADRA Lifesciences, Inc., Palo Alto, CA (US)

(72) Inventors: Mehmet Hakan Akpinar, Istanbul (TR); Corrado Tamburino, Sant' Agata Li Battiati (IT); Antonio Colombo, Milano (IT); Flavio Airoldi, San Donato Milanese (IT); Carlo Brigouri, Naples (IT); Federico De Marco, Milano (IT); Marwan Berrada Sounni, Los Gatos, CA (US); Rafal Wojciech Lopallo, Naples (IT); Scott Edward Parazynski, Houston, TX (US)

(73) Assignee: SQUADRA Lifesciences, Inc., Palo Alto, CA (US)

(21) Appl. No.: 18/077,999

(22) Filed: Dec. 8, 2022

Related U.S. Application Data
(63) Continuation of application No. PCT/US21/39160, filed on Jun. 25, 2021.
(60) Provisional application No. 63/044,279, filed on Jun. 25, 2020.

(51) Int. Cl. A61B 17/12; A61B 34/10; A61B 90/00; A61B 5/107

(52) U.S. Cl. CPC A61B 17/12122; A61B 17/12145; A61B 17/1215; A61B 34/10; A61B 90/39; A61B 5/1076; A61B 5/1079; A61B 2017/12086; A61B 2017/00022

(57) ABSTRACT

A device for occluding a patient's left atrial appendage (LAA) device includes a self-expanding closure disc and an anchor. The self-expanding closure disc has an atrial side, an appendage side, and a peripheral edge configured to engage tissue on both an atrial side and an appendage side of an opening between the left atrium and the left atrial appendage. The self-expanding anchor extends from the appendage side of the self-expanding closure disc, and an annular sealing skirt extends about at least a portion of the appendage side of the self-expanding closure disc. the annular sealing skirt is more compliant than the self-expanding closure disc to seal over the left appendage side of the opening after implantation of the device in the LAA.

### CROSS-REFERENCE TO RELATED APPLICATIONS

This application is a continuation of PCT Application No. PCT/US2021/039160 (Attorney Docket No. 55631-706.601), filed Jun. 25, 2021, which claims the benefit of U.S. provisional patent application 63/044,279 (Attorney Docket No. 55631-706.101), filed on Jun. 25, 2020, the full disclosure of which is incorporated herein by reference.

### BACKGROUND OF THE INVENTION

### 1. Field of the Invention

The present application relates generally to medical devices and methods and, more particularly, to devices and methods for closing a left atrial appendage in a patient's heart.

Atrial fibrillation (AF) is the most common arrhythmia and occurrences of this are expanding worldwide. The most significant complication of AF is systemic thromboembolism, particularly stroke. Blackshear et al. Ann Thorac Surg. (1996) 61(2):755-9 indicated that the left atrial appendage (LAA) is the most common site of thrombus formation, accounting for 91% of thrombi in patients with nonvalvular atrial fibrillation and 57% of thrombi in patients with valvular AF related to rheumatic heart disease.

The LAA is a small, ear-shaped sac in the muscle wall of the left atrium. The normal heart contracts with each heartbeat, and the blood in the left atrium and LAA is squeezed out of the left atrium into the left ventricle. When AF occurs, the impulses that control the heartbeat begin at the same time and spread through the atria. The fast and chaotic impulses do not give the atria time to contract and/or effectively squeeze blood into the ventricles. Because the LAA is a little pouch, blood collects there and can form clots in the LAA and atria. When blood clots are pumped out of the heart, they can cause a stroke.

Oral anticoagulation is the most common therapy for reducing the risk of atrial fibrillation-associated thromboembolic stroke and can be individualized according to a patient's stroke-risk and the presence or absence of comorbidities. Oral coagulation is not always effective, and the need to anti-coagulate for extended periods carries its own risk of hemorrhagic stroke. Thus, there is thus a need for alternative treatment strategies that eliminate or at least reduce the need for anti-coagulation.

Proposed alternatives to anti-coagulation for LAA treatment include open-heart and minimally invasive procedures for removing, occluding, and/or sealing the LAA from the patient's left atrium, such as LAA excision, endocardial and epicardial suturing, using epicardial clip devices, other percutaneous closure techniques, and the like.

Of interest to the present invention, LAA occluding devices having anchoring portions and sealing portions have been proposed for intravascular, transthoracic, and transapical delivery. The anchoring portion may comprise a self-expanding cage or other structure which implants in the interior of the LAA and a flexible polymeric or other seal which covers and is intended to seal the opening into the LAA.

While a very promising approach, such LAA occlusion devices may suffer from shortcomings, such as the sealing portion having a poor fit and/or edge seal with the opening of the LAA which can allow the release of emboli from the LAA after implantation. Thus, it would be desirable to provide alternative LAA occlusion devices which provide for an improved fit and/or seal with the LAA opening to better inhibit the loss of emboli from the LAA. At least some of these objectives will be met by the inventions described and claimed herein.

### 2. Listing of the Background Art

Relevant patents and publications include EP2716237; CN2021/143640; EP3241498; WO2018/199854; U.S. Pat. Nos. 7,056,294; 6,152,144; US2017/0224354; and U.S. Pat. No. 8,034,061.

### SUMMARY OF THE INVENTION

In a first aspect, the present invention provides a device for occluding a patient's left atrial appendage (LAA). The device comprises a self-expanding closure disc and a self-expanding anchor. The self-expanding closure disc has an atrial side, an appendage side, and a peripheral edge configured to engage tissue on both an atrial side and an appendage side of the opening. The self-expanding anchor extends from the appendage side of the self-expanding closure disc, and an annular sealing skirt or disc extends about at least a portion of the appendage side of the self-expanding closure disc. The annular sealing skirt or disc is more compliant than the self-expanding closure disc to provide enhanced sealing over the left appendage side of the opening after implantation of the device in the LAA (enhanced relative to sealing in the absence of the annular sealing skirt).

In preferred instances, the self-expanding closure disc comprises a woven wire frame, e.g. formed from nitinol or other superelastic metal wires.

In preferred instances, an annular sealing skirt may comprise a woven wire frame, e.g. woven from nitinol or other superelastic metal wires. where the wires of the annular sealing skirt are less stiff than the wires of the wires of the self-expanding closure disc and/or wherein the wires of the annular sealing skirt are woven less densely than the wires of the wires of the self-expanding closure disc. In particular examples, the wires of the annular sealing skirt may comprise extensions of the wires of the self-expanding closure disc, e.g. the wire extensions of the self-expanding closure disc are formed in a serpentine, zig-zag or other similar meandering or reversing pattern which extends beyond a peripheral edge of the self-expanding closure disc. Alternatively, the annular sealing skirt may be attached with a separate wire.

In preferred instances, an annular sealing disc may comprise a woven wire frame, e.g. woven from nitinol or other superelastic metal wires. where the wires of the annular sealing disc are less stiff than the wires of the wires of the self-expanding closure disc and/or wherein the wires of the annular sealing disc are woven less densely than the wires of the wires of the self-expanding closure disc. In particular examples, the annular sealing disc will caver most or all of the left appendage face of the self-expanding closure disc and may be connected by interweaving or use of separate attachment means.

In preferred instances, the devices of the present invention may further comprise a blood barrier material extending across the self-expanding closure disc and covering an appendage side of the skirt. A preferred blood barrier material comprises expanded polytetrafluoroethylene (ePTFE). In specific examples, the closure disc may comprise a braided body, typically a metal wire braid, comprising shape-memory wires, and a port or fenestration is created during shape-memory forming process to locate and attach the one-way valve inside the disc frames and seal with the blood barrier material, such as ePTFE. Typically, while loading the device into a delivery sheath, a guidewire will be located though the one-way valve so that the guidewire is pre-positioned to advance the suction catheter after implantation of the devices. Aspiration applied through the suction catheter is confirmed by either observing the cessation of blood flow through the suction catheter or pulling or by gently pulling on the implanted device to directly confirm that the device is being held in place by the vacuum force. The catheter and the guidewire will be removed and the LAA may be closed without any residual blood or very little inside the LAA pouch.

In some instances, the delivery catheters and chiefs of the present invention may comprise a pusher cable having a coupling element at its distal land, such as a magnetic coupling element, and in particular an electromagnetic coupling element. The hub on the self-expanding closure disc will also have a hub which releasably interconnects with the hub on the pusher cable. For example, when the coupling element on the pusher cable is an electromagnetic component, the hub on the self-expanding closure disc will typically be a ferromagnetic material. In this way, the pusher cable is useful both for delivering and releasing the self-expanding closure disc at the capital LAA and optionally for retrieving the closure disc during initial deployment or later.

In preferred instances, the self-expanding closure disc may comprise an atrial disc and an appendage disc. The atrial disc and the appendage disc may have the same or different peripheral geometries. For example, the atrial disc may be rounder, and the appendage disc may be more oval, or vice versa, depending on the patient's LAA anatomy, for example as determined using the sizing tool as described elsewhere herein. In such instances, the one-way valve may be held between the atrial disc and the appendage disc.

In preferred instances, the annular sealing skirt may comprise wire extensions of the appendage disc.

In preferred instances, the self-expanding anchor may comprise at least a first pair of everting wires configured to open in a plane normal to a plane of the self-expanding closure disc often further comprising at least a second (one additional) pair of everting wires configured to open in a plane normal to a plane of the self-expanding closure disc. In some instances, the at least second (one additional) pair of everting wires is coplanar with the at least one pair of everting wires. In other instances, the second (at least one additional) pair of everting wires is out of planar alignment with the first (at least one) pair of everting wires. In a particularly preferred arrangement, the first at pair of everting wires is orthogonal to both the self-expanding closure disc and the second pair of everting wires.

In preferred instances, the self-expanding closure disc has an oval periphery.

In preferred instances, the closure discs of the present invention further comprise a port formed through the self-expanding closure disc and configured to allow aspiration, perfusion of contrast medium, and/or introduction of occlusive materials through the self-expanding closure.

Aspiration of the LAA pouch may be performed with a suction catheter is through a one-way valve, preferably a double check-valve made of soft biocompatible materials (implant grade) located on both sides of closure discs.

In preferred instances, the self-expanding closure and the self-expanding anchor may be detachably secured to each other.

In a second aspect, the present invention provides a system comprising any of the device designs described previously and elsewhere herein, in combination with one or more additional self-expanding closures, wherein at least two self-expanding closures have a size, shape, or other characteristics different from each other to allow a selection to be made based on patient anatomy.

In preferred instances, the systems of the present invention may further comprise one or more additional self-expanding anchors, where at least two self-expanding anchors have size, shape, or other characteristics different from each other to allow a selection to be made based on patient anatomy.

In a third aspect, the present invention provides a method for occluding a left atrial appendage (LAA) of a patient. The method comprises providing a plurality of self-expanding closure discs, each disc having an atrial side, an appendage side, and a peripheral edge configured to engage tissue on both the atrial side and the appendage side of the opening, and providing a plurality of self-expanding anchor configured to be attached to the appendage side of the self-expanding closure disc. A size, shape, or other anatomical characteristic of the patient's LA is determined, and one of the plurality of self-expanding closure discs is selected based on the determined size, shape, or other anatomical characteristic of the patient's LAA. One of the plurality of self-expanding anchors is also selected based on the determined size, shape, or other anatomical characteristic, and the selected self-expanding closure disc and the selected self-expanding anchor are assembled to form an implantable assembly. The implantable assembly having specific size and/or shape characteristics chosen based upon the patient's anatomy may then be implanted in the patient's LAA.

In preferred instances, the size, shape, or other anatomical characteristic of the patient's LAA may be selected by introducing a sizing device into the LAA, expanding the sizing device in three dimensions within the LAA to span the depth of the LAA, and the width of the LAA in at least two directions, and imaging the expanded sizing device to determine both size and shape of the LAA. Typically, the width will be spanned in at least two directions normal to each other and to a longitudinal axis of the LAA. For example, the sizing device may be the one described elsewhere in this disclosure.

In a fourth aspect, the present invention provides a method for occluding a patient's left atrial appendage (LAA). The LAA closure device as in any one of embodiments described herein is provided. The closure disc is introduced in a constrained configuration and allowed to self-expand to span the opening between the left atrium and the left atrial appendage to engage tissue on both an atrial side and an appendage side of the opening. The anchor is also introduced in a constrained configuration and allowed to self-expand within the interior of the LAA to immobilize the closure disc over the LAA opening, where the annular sealing skirt extends about at least a portion of the appendage side of the self-expanding closure disc.

In preferred instances, the methods for occluding the LAA of the present invention further comprise aspirating blood from the LAA though a port in the closure disc and/or introducing clot-inducing materials into the LAA though a port in the closure disc.

In preferred instances, self-expanding the anchor comprises expanding a plurality of everting wires which laterally stretch the walls of the LAA to hold the anchor and closure disc in place.

In a fifth aspect, the present invention provides a device for sizing a patient's LAA). The device comprises a shaft and a sizing head attached to a distal end of the shaft, where the sizing head is configured to expand within an interior of the LAA. Markers on the sizing head conform to an interior wall of the LAA when the sizing head is expanded therein, where the markers distribute over the interior wall sufficiently to delineate the size and shape of the wall under external imaging, e.g. fluoroscopic, ultrasonic, such as transesophageal echocardiography, and the like.

In some examples, the sealing discs may comprise one or more physiologic sensors for measuring pressure, temperature, displacement, orientation, compression, or the like. In one instance, the sensor may be a pressure or force sensor disposed on an atrial face of the sealing disc to measure, for example, wall pressure being exerted at the mouth of the capital LAA.

In some examples, all or a portion of the sealing skirts of discs may be coated with one or more anti-thrombin substances, such as heparins, anticoagulants, and the like.

In preferred instances, the shaft may be configured to be advanced intravascularly, trans-thoracically, or transapically into the patient's left atrium and left atrial appendage.

In preferred instances, the sizing head may comprise at least three everting wires, preferably at least four everting wires, with the markers spaced apart along a length of each wire.

In preferred instances, the sizing head may comprise a membrane cover.

In further and alternative aspects, sizing devices comprise features that enable measuring both the depth of the LAA as well as the opening from multiple perspectives at one time and make it visible under fluoroscopy.

In specific instances, sizing head comprises three or more everting (curved) pre-shaped shape memory (nitinol) wires with radiopaque or echogenic markers located with a spacing discernable under fluoroscopy and/or ultrasonography which provides a scale to measure the desired length and distance precisely to choose the correct size of the device for closure or elimination of the LAA. The use of curved, everting wires allows the width of the sizing had to be adjusted by pushing the distal end of the sizing head against the closed end of the LAA. In this way, the edges and/or tips of the wires can be expanded to span the entire interior volume of the LAA. Such pushing and engagement of the wires can also provide an estimation of the elastic enlargement of the LAA tissue.

### BRIEF DESCRIPTION OF THE DRAWINGS

FIG. 1 is a schematic illustration of an exemplary occlusion device constructed in accordance with the principles of the invention shown deployed in a patient's left atrial appendage (LAA).

FIG. 1A is a plan view of the LAA occlusion device in FIG. 1 showing a preferred oval periphery.

FIGS. 1B-1C show details of regions 1B-1B, 1C-1C, and 1D-1D of FIG. 1.

FIG. 2 is a schematic illustration of a second exemplary occlusion device constructed in accordance with the principles of the invention shown with an annular sealing skirt about the periphery of a closure disc.

FIG. 2A shows a detail of the annular sealing skirt in region 2A-2A of FIG. 1.

FIGS. 2B and 2C are top and side views of a third exemplary occlusion device constructed in accordance with the principles of the invention shown with a third, sealing disc secured over the inside face a closure disc with a connecting wire.

FIGS. 2D and 2E are top and side views of a fourth exemplary occlusion device constructed in accordance with the principles of the invention shown with a third, sealing disc secured over the inside face a closure disc by interweaving wires of the discs.

FIG. 3 is an enlarged view of a portion of FIG. 1 showing a skeleton wire passing a sealing port in the closure disk.

FIG. 4 illustrates a system for delivering the LAA occlusion devices of the present invention to a patient's LAA.

FIG. 4A is an enlarged view of a pusher cable of the delivery system of FIG. 4.

FIG. 5 is a schematic illustration of a preferred embodiment of a sizing device of the present invention.

FIG. 6 is a schematic illustration of an alternative preferred embodiment of a sizing device of the present invention.

FIGS. 7 and 7A illustrate an exemplary occlusion device constructed in accordance with the principles of the invention with wires or coils positioned circumferentially over a peripheral groove which has a high coefficient of friction and hydrophobic properties and which seats on a shoulder in the transition between the atrium and a sac of the appendage.

FIGS. 8A and 8B illustrate an exemplary occlusion device constructed in accordance with the principles of the invention with a pusher cable magnetically coupled to a hub on the occlusion device.

FIG. 9 illustrates an exemplary occlusion device constructed in accordance with the principles of the invention having a force sensor on a face thereof.

### DETAILED DESCRIPTION OF THE INVENTION

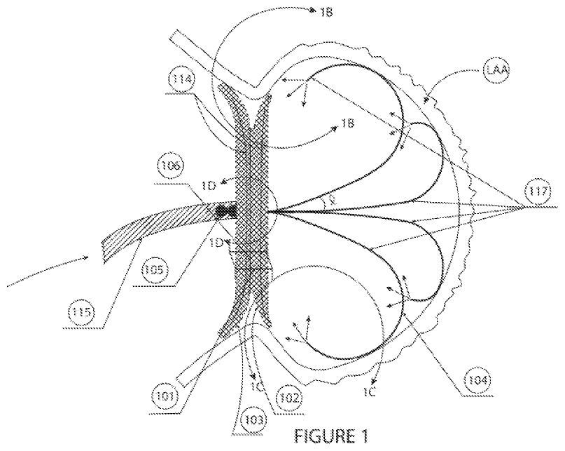

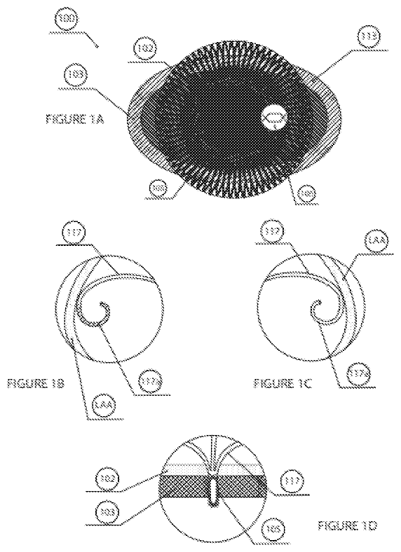

As seen in FIG. 1, an exemplary left atrial appendage (LAA) closure device 100 comprises a braided body 101 formed from nitinol (nickel-titanium alloy) or other superelastic metallic wires. The braided body 101 is formed into at least one closure disc, typically being formed into at least an outer or “atrial” closure disc 102 and an inner or “appendage” closure disc 103 and has a shape-memory which is self-expandable from a low profile delivery configuration to an expanded configuration (shown in FIG. 1) sized and shaped to cover an opening O of the LAA when e closure device is implanted. The braided body 101 is typically covered or laminated with a blood barrier material 114, sometimes referred to as a “patch” material, such as expanded polytetrafluoroethylene (ePTFE) or other material of the type used in vascular grafts, typically having one layer on or over the atrial closure disc 102 and one layer on or over the appendage closure disc 103. A self-expanding anchor structure comprising three or more curved or everting anchoring wires extends in a normal direction from the appendage side of the braided body 101, and a hub connection 105 for connection to a delivery cable 115 of a delivery sheath system is disposed on an atrial side of the braided body 101. The hub may be configured for threaded attachment, magnetic attachment, snap-on attachment, and other conventional attachable/detachable connection structures well known in the intravascular delivery arts. A one-way or “check-valve” assembly typically extends through the braiding mesh, and the delivery cable may have a lumen to deliver contrast media into the LAA pouch to examine the LAA anatomy under fluoroscopy during an implantation procedure. In FIG. 1, arrows on the tips of the wires 117 indicate that the contrast or other media is being delivered through hollow wires.

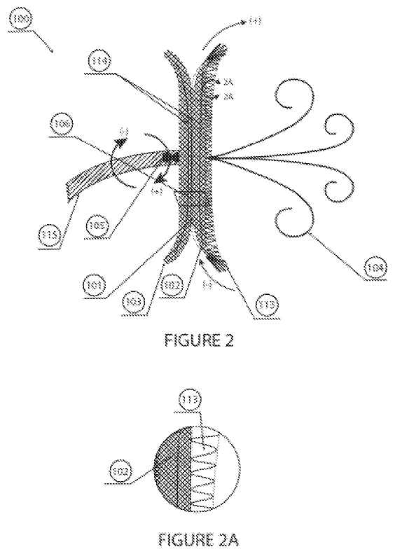

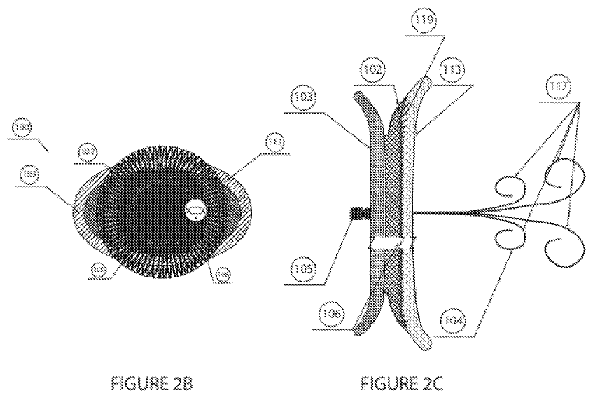

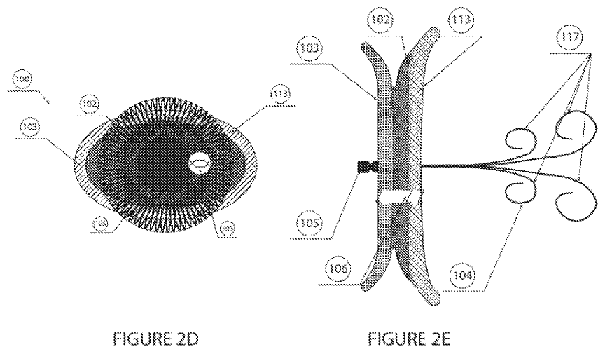

FIG. 1A is a plan view of the braided body 100 showing a preferred oval periphery and a preferred configuration with an outer annular edge of the atrial closure disc 103 extending beyond the outer periphery of the appendage disc 102. A preferred annular sealing skirt 113 (see also FIG. 2) extends beyond the outer periphery of the atrial closure disc 103 to provide enhanced sealing of the LAA pouch.

FIGS. 1B and 1C illustrate optional fully everted tip 117a which provide atraumatic engagement for wires 117 as the anchor assembly is expanded in the atrial pouch of the LAA.

FIG. 1D is a detailed view of the hub 105 which detachably connects to a distal end of the delivery sheath 115.

FIGS. 2 and 2A are schematic illustrations of a preferred embodiment of the braided body 100 similar to that shown in FIG. 1 but further including a skirt or sealing layer 113 surrounding and extending peripherally outwardly from the inner, appendage disc 102.

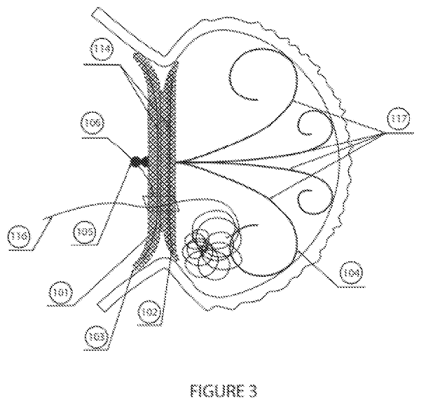

In an alternative embodiment, as shown in FIG. 3, a skeleton wire 116 may be introduced through the one-way valve 106 to form a sealing layer connected to the inner appendage disc 102.

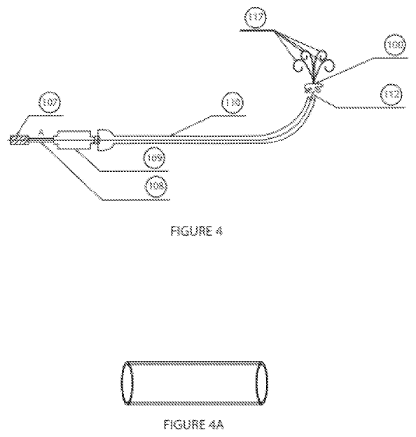

As shown in FIG. 4, a delivery system of the invention device includes a sheath 110, such as a Mullin's sheath, a pusher cable handle 107, a pusher cable main body 108 (also shown in FIG. 4A), a loader 109, and a coupler 112 which detachably engages the hub 105 (FIGS. 1 and 2) on the braided body 100.

Referring to FIGS. 1 to 4, system of the invention comprise a braided body comprising or consisting of a shape-memory, self-expandable metallic wires forming outer (atrial) and inner (appendage) sealing disc of the braided body, an external disc of the braided body, three or more anchoring spiral wires, a hub connection for delivery system, the check-valve(s) included in braiding mesh and a third internal disc for rapid thrombus formation and endothelization in LAA pouch.

FIGS. 2B and 2C are top and side views of a third exemplary occlusion device 100 constructed in accordance with the principles of the invention shown with a sealing skirt 113 secured over the inside face a closure disc with a connecting wire 119. The connecting wire structure 119 between inner appendage disc 102 and annular sealing skirt 113 which provides a transition part from stiff and dense structure to soft and less dense structure.

FIGS. 2D and 2E are top and side views of a fourth exemplary occlusion device constructed in accordance with the principles of the invention shown with a third, sealing disc 113 secured over the inside face the inner appendage disc 102 by interweaving wires of the discs. The wire density in the sealing disc will typically be lower than the wire density in the inner appendage disc to provide a softer structure with improved sealing characteristics.

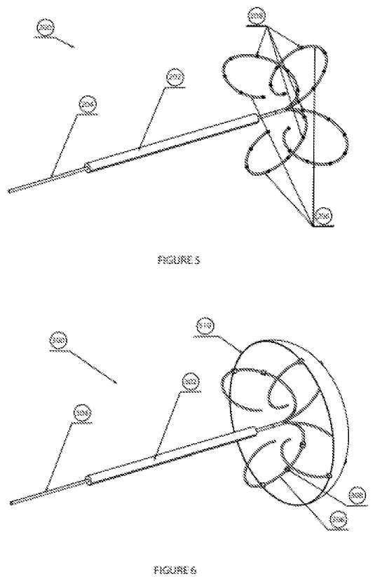

FIG. 5 is a schematic illustration of a preferred embodiment of a LAA sizing device 200 of the present invention showing four pre-shaped nitinol wires 206 deployed from a sheath 202 using a pusher wire 204. Imaging markers, typically fluoroscopically or ultrasonically opaque markers, are spaced apart along the length of each wire so that the markers can be imaged when the wires are expanded in the pouch of a patient's LAA. By observing and measuring the spacing of the markers under external imaging, the size and shape of the LAA pouch can be determined, allowing selection of appropriate sealing disc and anchor components for the implant.

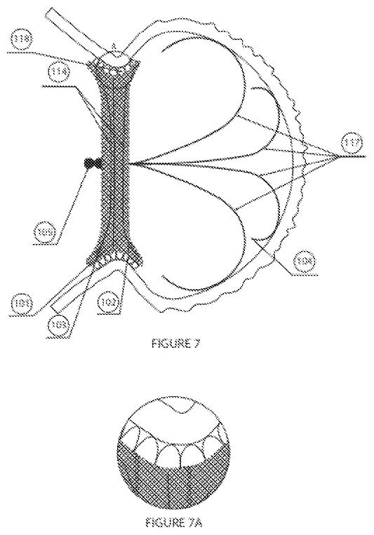

FIG. 6 is a schematic illustration of an alternative preferred embodiment of a LAA sizing device 300 of the present invention including pre-shaped nitinol wires 306 deployed from a sheath 302 using a pusher wire 304. Markers 308 similar to those of sizing device 200 may be provided on the wires, and a generally hemispherical membrane 310 may be positioned over the distal portions of the wires 306 to enhance conformance to the LAA pouch. Markers (not shown) may optionally be placed on the membrane in addition or as an alternative to those on the wires.

In an exemplary LAA occlude delivery protocol, the delivery sheath 110 of introducer system is placed into a femoral vein over a pre-placed 0.035″ guidewire and is advanced through the venous access site of the body passing from vena cava inferior, right atrium, intra-atrial septum after the septostomy and the left atrium until a tip of the sheath is in the left atrial appendage (LAA) area.

Initially, the sizing device 300 is loaded through a loader 109 and flushed with a saline solution to eliminate residual air bubbles which might cause air-embolization. The sizing device is connected to a pusher cable 304 to mount and release the device at the desired location. The sizing device is pushed by the help of pusher cable 304 through the delivery sheath 302 and the device is opened into the LAA pouch. The sizing device that consists of three or more spiral pre-shaped nitinol wires 306 with radiopaque markers 308 located with a specific distance from each other to have enough distance range to see under fluoroscopy, transesophageal echocardiography, or the like, and measure the desired length and distance precisely to choose the correct size of the invention device for closure or elimination of the LAA. The diameter of the sizing device will be increased by pushing the sizing device shaft 302 through the delivery sheath 302 and decreased by pulling the sizing device shaft 302 backward. This can be done until the edge of spiral pre-shaped nitinol wires 306 touch to the orifice of the LAA. Furthermore, the pushing might be done to investigate the elastic enlargement of the LAA tissue. The sizing device might also have an external membrane 310 to see the total geometry of the LAA orifice and pouch to have a better understanding of how the occlude will conform to the anatomy of the LAA. The sizing device is pulled back to the delivery sheath 302 and removed after providing to the anatomy characteristics.

Then, a suitable size of LAA closure or elimination device of the invention is loaded to a loader 109 and flushed with a saline solution to eliminate the risk of residual air bubbles which might cause air-embolization. The loader 109 is connected to the delivery sheath 110 or 302 with a male-female Luer locking mechanism. The LAA closure or elimination device is connected through a connection part 111 to a pusher cable 108 which is located in a delivery sheath 110 with a hub connection 105 to mount and release the device at the desired location rotating the screw on and off through a pusher cable handle 107. The LAA closure or elimination device is pushed by the help of pusher cable 108 through the delivery sheath 110 and the device is opened into the LAA pouch. Aspiration of the LAA pouch with a suction catheter is performed through a double check-valves) 106 located on both sides of the LAA closure or elimination device discs 102, 103. The check-valve(s) 106 which are made of soft biocompatible materials (implant grade) will be located inside the braided body. The braiding is done according to the geometry and the fenestration is created during shape-memory process to locate and fix the check-valve(s) 106 inside the disc frames 102, 103 and seal with the patch material 114 such as e-PTFE and similar to keep it blood tide after the aspiration is applied. While loading the invention device to the loader 109 to advance inside the delivery sheath 110 with a guidewire will be left inside this check-valve(s) 106 to be able to advance the suction catheter afterward or at the same time since the high pressure is on LA side and vacuum on the LAA pouch. The check-valve(s) 106 are locked from outside to inside where enough vacuum force is obtained. The device anchors precisely to the LAA orifice.

The aspiration applied through the suction catheter is checked by either confirmation of no blood coming from the suction catheter or pulling the pusher cable 108 gently that the device disc does not move because of the vacuum force. The device of the invention has a double check-valve(s) 106 on both discs 102, 103 to create the sealing between the suction catheter and the LAA elimination device and keep the vacuum force after retrieving the suction catheter from the LAA elimination device. This valve(s) 106 kept open with a preloaded guidewire to advance the suction catheter and after applying the aspiration and creating the vacuum force until the internal volume of the LAA pouch is empty to a certain point that the aspiration cannot be performed anymore which create the highest possible vacuum force and ensure that no residual shunt or leak is existing since the vacuum force will decrease and further aspiration can be performed because of the leak or shunt. The vacuum force will suck the device discs 102, 103 towards the orifice walls and create an additional force to anchor the device to the LAA pouch more precisely. The check valve(s) 106 for aspiration of the LAA pouch can be designed and manufactured as the inside the device hub 105a and the anchoring wires 104a can be made from tubing which is connected to the hub 105a to do the aspiration from the hub 105a and the anchoring tubes 104a attached to the hub 105a. Furthermore, the hub 105a is connected to a hollow shaft on the device from 105a to 104c and the hollow shaft pusher cable 108 a which can be used as an aspiration lumen for the device system. This hollow shaft system can also be used for injecting contrast media to check the internal lumen of the LAA pouch is sealed with the device discs 102, 103 and after the device location and the sealing is completed further aspiration can be applied to empty the pouch to create a vacuum force to increase the anchoring and stability of the device.

The catheter and the guidewire will be removed and the LAA will be closed without any residual blood or very little inside the LAA pouch. The suction and the vacuum power will make the LAA pouch shrink over the device internal disc 102 and create a sealing of the device which can be proven with the suspension of the vacuum power inside the LAA pouch. If there is any residual shunt the vacuum will decrease in the pouch and the suction catheter can suck continuously. Furthermore, the vacuum power would be an additional force to keep the LAA elimination system in place together with the anchoring spiral nitinol wires 104.

One embodiment of the invention device consists of a larger LA external disc 103 with rigid construction and a softer internal disc 102 inside the LAA pouch to conform according to the anatomy after the suction force is applied. When the internal disc 102 is surrounded by the LAA tissue, the sealing will be created and the vacuum power will be obtained during aspiration of the blood inside the LAA pouch. The internal disc 102 might include a patch material 114 made of PTFE or similar materials.

The external disc 103 facing to the left atrium has a dense wire concentration to have a more rigid structure to close the LAA orifice when in contact with the surrounding tissue around the LAA orifice, on the other hand, the internal disc 102 inside the LAA pouch is softer and compliant compared to the external disc 103 and less dense in wire 101 concentration.

The area of the larger external disc 103 which is bigger than the LAA orifice will be in contact with the surrounding tissue and creates a sealing surface because the vacuum power will suck the disc towards the LAA pouch. The smaller internal disc 102 which is softer and can adapt to the anatomy uniformly will cover all internal surfaces of the LAA pouch and apply the vacuum force to the tissue which shrinks around the soft internal disc 102 to create a better sealing. The external disc 103 can be rounder and the internal disc 102 can be more oval or reverse wise with the sizes and anatomy changes of the patient. Also, the external disc 103 might include a patch material 114 made of PTFE or similar materials.

Another embodiment of the invention device includes three or more adjustable and replaceable anchoring spiral wires 104 which might be on the same plane or might have angulation between them to create better anchoring and conform to the anatomy to create gentle but sufficient anchoring points and contact locations while not inflating the anatomy. The wires are stretching the pouch horizontally to deflate the pouch volume and create more friction between the anchoring spiral wires 104 and the internal surface of the LAA pouch. The tip of the anchoring wires 104b might be soft tip J type guidewire distal end, coiled and formed to be less traumatic and more conforming to the anatomy of the surrounding tissue. Furthermore, these soft tip J type design can be made of radiopaque material like platinum-iridium to be visualized under fluoroscopy for safer and efficient implantation. These anchoring wires 104 can also have an external polymer cover, coating, or sleeves 104c to keep the metallic parts inside to protect the whole structure inside to eliminate the risk of fracture of the metallic part. This polymer sleeve 104c can be hydrophobic coated to have better friction with the LAA pouch internal surface for better anchoring.

After sizing the LAA orifice and the depth, the device can have two or more steps alternative anchoring spiral wires 104 either on the same plane or different angulation to create 3D geometry inside the LAA pouch, the spiral wires 104 with following features such as shallow depth, medium depth and deep depth which can be replaceable so that after measuring the dimensions of the LAA the physician can load the suitable size of the anchoring spiral wires 104 to the device to achieve maximum safety for the right size of device fixation to the LAA anatomy. Nevertheless, the anchoring wires 104 can be advanced in one step with three different depths at the same time to anchor the device safely in complex anatomies. Two or more steps can be also created with anchoring spiral wires 104 with telescopic design and tubing pushing and locking of the device is placed at the LAA orifice. The physician can push the anchoring spiral wire 104 inside the LAA pouch as much as he is satisfied with the location and anchoring force afterward can lock anchoring spiral wires 104 the telescopic design to fix the device to anchor.

Another embodiment of the invention device includes a third internal disc 113, very soft compliant and larger than the internal disc 102 by diameter and volume creates a sealing layer without extra stretching the anatomy and using oversized devices, this extra soft and compliant layer creates a soft contact with every point of the surrounding tissue. The third sealing layer (disc) 113 can be consist of a soft braided thinner shape memory alloy wires from the original disc wire diameter which might have thrombogenic filaments to achieve instant closure and rapid endothelization. The third sealing layer (disc) 113 can be also coated with a hydrophobic surface to be attached to the tissue surface and creates an instant sealing with rapid endothelization. Furthermore, these two features can be combined to enhance the safety and efficiency of the LAA elimination device.

The further embodiment of the invention device describes the components of the device that includes an internal and external disc 102, 103 consisting of self-expandable and shape memory braided mesh made of nitinol wires 101 or similar materials having different braiding patterns to obtain different characteristics according to the intended use and area, a patch material 114 made of PTFE or similar materials for both discs 102, 103, three or more adjustable and replaceable anchoring spiral wires 104 made of nitinol or similar materials, the check-valve(s) 106 made of soft biocompatible materials (implant grade) and curtain layer with microfibers 113 made of a medical-grade hydrophobic material, a skeleton wire 116 to form sealing layer connected to the internal disc 102 and a hollow shaft delivery cable 115 which allows the contrast media transition into the LAA pouch to examine the LAA anatomy under fluoroscopy.

In some embodiments of the invention, the sizing device includes three or more pre-shaped nitinol or similar shape-memory wires 306 with radiopaque markers 308 made of platinum-iridium or similar materials, a device shaft 302 and an external membrane 120 made of PET, PTFE or similar materials.

FIGS. 7 and 7A illustrate an exemplary occlusion device constructed in accordance with the principles of the invention with wires or coils positioned circumferentially over a peripheral groove which has high coefficient of friction and hydrophobic properties and which seats on a shoulder in the transition between the atrium and a sac of the appendage. In some embodiments, the high coefficient of friction and hydrophobic surface together increase the sealing rate and enhance anchoring.

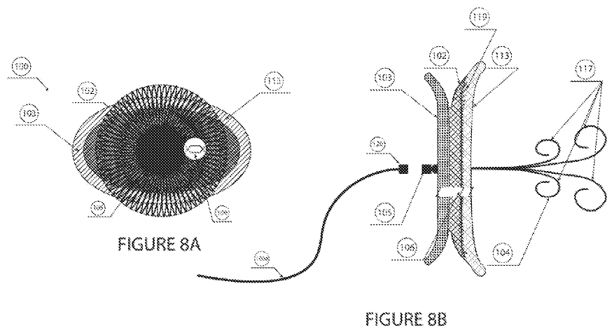

FIGS. 8 and 8A illustrate an exemplary occlusion device 100 constructed in accordance with the principles of the invention with a pusher cable 108a having an electromagnetic distal coupling element 120 which couples to a ferromagnetic hub 105 on the occlusion device 100. The pusher cable 108a can be incorporated into a delivery catheter or sheath, and the electromagnetic distal coupling element 120 can be energized and de-energized to hold and release the ferromagnetic hub 105. Such a detachment system is useful to load, push, and release the occlusion device during deliver and deployment.

The connection is created and maintained during delivery and positioning within the LAA and released by turning on the electromagnetic field on the pusher and when the device is at the desired location.

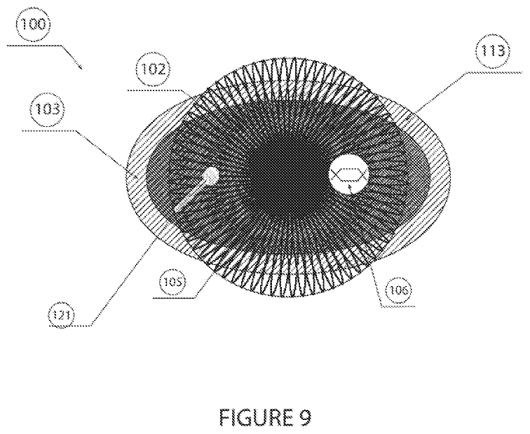

FIG. 9 illustrates an exemplary occlusion device constructed in accordance with the principles of the invention having a force sensor 121 on a face thereof. The force sensing resistor 121 is located on the left atrial disc of the LAA device provides data collection from the atrial side of the device such as blood pressure, residual leak, pressure gradient between the two sides of the device to provide additional critical information to the physician who performs the intervention.

While the above is a complete description of the preferred embodiments of the invention, various alternatives, modifications, and equivalents may be used. Therefore, the above description should not be taken as limiting the scope of the invention which is defined by the appended claims.

What is claimed is:

1. A device for occluding a patient's left atrial appendage (LAA) having an opening between the left atrium and the left atrial appendage, said device comprising:
a self-expanding closure disc having an atrial side, an appendage side, and a peripheral edge configured to engage tissue on both the atrial side and the appendage side of the opening;
a self-expanding anchor extending from the appendage side of the self-expanding closure disc; and
an annular sealing skirt or disc extending about at least a portion of the appendage side of the self-expanding closure disc, wherein the annular sealing skirt or disc is more compliant than the self-expanding closure disc to provide enhanced sealing over the left appendage side of the opening after implantation of the device in the LAA.

2. The device of claim 1, wherein the self-expanding closure disc comprises a woven wire frame.

3. The device of claim 2, wherein the annular sealing skirt or disc comprises a woven wire frame and wherein the wires of the annular sealing skirt are less stiff than the wires of the wires of the self-expanding closure disc and/or wherein the wires of the annular sealing skirt are woven less densely than the wires of the wires of the self-expanding closure disc.

4. The device of claim 3, wherein the wires of the annular sealing skirt or disc comprise extensions of the wires of the self-expanding closure disc.

5. The device of claim 4, wherein the wire extensions of the self-expanding closure disc are formed in a serpentine or zig-zag pattern which extends beyond a peripheral edge of the self-expanding closure disc.

6. The device of claim 1, further comprising a blood barrier material extending across the self-expanding closure disc and covering an appendage side of the sealing skirt or disc.

7. The device of claim 1, wherein self-expanding closure disc comprises an atrial disc and an appendage disc.

8. The device of claim 7, wherein the annular sealing skirt comprises wire extensions of the appendage disc.

9. The device of claim 7, wherein the annular sealing disc is secured to the self-expanding closure disc with connecting wire.

10. The device of claim 1, wherein the self-expanding anchor comprises at least one pair of everting wires configured to open in a plane normal to a plane of the self-expanding closure disc.

11. The device of claim 9, further comprising at least one additional pair of everting wires configured to open in a plane normal to a plane of the self-expanding closure disc.

12. The device of claim 11, wherein the at least one additional pair of everting wires is coplanar with the at least one pair of everting wires.

13. The device of claim 11, wherein the at least one additional pair of everting wires is out of planar alignment with the at least one pair of everting wires.

14. The device of claim 13, wherein the at least one additional pair of everting wires is orthogonal to both the self-expanding closure disc and the at least one pair of everting wires.

15. The device of claim 1, wherein the self-expanding closure disc has an oval periphery.

16. The device claim 1, further comprising a port formed through the self-expanding closure disc and configured to allow aspiration, perfusion of contrast medium, and/or introduction of occlusive materials through the self-expanding closure.

17. The device of claim 1, wherein the self-expanding closure disc and the self-expanding anchor are detachably secured to each other.

18. The device of claim 1, wherein further comprising a sensor on an atrial face of the self-expanding closure.

19. A system comprising:
the device of any one of claim 1; and
a pusher cable configured to magnetically couple to the self-expanding closure.

20. A system comprising:
the device of claim 1; and
one or more additional self-expanding closures, wherein at least two self-expanding closures have size, shape, or other characteristics different from each other to allow a selection to be made based on patient anatomy.

21. The system of claim 20, further comprising one or more additional self-expanding anchors, wherein at least two self-expanding anchors have size, shape, or other characteristics different from each other to allow a selection to be made based on patient anatomy.

22. A method for occluding a left atrial appendage (LAA) of a patient, said method comprising:
providing a plurality of self-expanding closure discs having an atrial side, an appendage side, and a peripheral edge configured to engage tissue on both the atrial side and the appendage side of the opening;
providing a plurality of self-expanding anchor configured to be attached to the appendage side of the self-expanding closure disc;
determining a size, shape, or other anatomical characteristic of the patient's LAA;
selecting one self-expanding closure disc from the plurality of self-expanding closure discs based on the determined size, shape, or other anatomical characteristic;
selecting one self-expanding anchor from the plurality of self-expanding anchors based on the determined size, shape, or other anatomical characteristic;
assembling the selected self-expanding closure disc and the selected self-expanding anchor to form an implantable assembly; and
implanting the implantable assembly in the patient's LAA.

23. A method for occluding a patient's left atrial appendage (LAA) having an opening between the left atrium and the left atrial appendage, said method comprising:
providing a LAA closure device as in any one of claim 1;
self-expanding the closure disc the atrial side to engage tissue on both an atrial side and an appendage side of the opening;
self-expanding the anchor within the interior of the LAA to immobilize the closure disc over the LAA opening, wherein the annular sealing skirt extends about at least a portion of the appendage side of the self-expanding closure disc.

24. A device for sizing a patient's left atrial appendage (LAA), said device comprising:
a shaft;
a sizing head attached to a distal end of the shaft and configured to expand within an interior of the LAA; and
markers on the sizing head which conform to an interior wall of the LAA when the sizing head is expanded therein and which distribute over the interior wall sufficiently to delineate the size and shape of the wall under external imaging.
